(19) United States
(12) Patent Application Publication
(10) Pub. No.: US 2008/0110783 A1
(43) Pub. Date: May 15, 2008

(54) Array of packages having indicators of non-visual properties of absorbent articles

(76) Inventors: Brian Kenneth Burgdorf, Norwood, OH (US); Gabriela Valerio Gonzalez, Cincinnati, OH (US); Jessica Lee Hansman, Cincinnati, OH (US); Jean Marie Knuth, Cincinnati, OH (US); Signe Christina Larson, Cincinnati, OH (US); Eduardo Enrique Souchon, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/595,636

(22) Filed: Nov. 10, 2006

(51) Int. Cl. A61B 19/00; B65D 85/00; B65D 73/00; A61B 19/02

(52) U.S. Cl. 206/440; 206/459.1; 206/494; 206/438

(57) ABSTRACT

An array of packages is provided wherein each package has non-visual property indicators. The non-visual property indicators can be any indicator suitable for visually communicating to a consumer, the non-visual property of the absorbent articles contained in a package, in relation to other absorbent articles contained in other packages within an array of packages. The packages can comprise an interior surface and an exterior surface, the interior surface defining an interior space. Absorbent articles can be disposed within the interior space and the exterior surface can include non-visual property indicators.

### FIELD OF THE INVENTION

This invention relates to arrays of packages, particularly wherein the packages contain absorbent articles.

### BACKGROUND OF THE INVENTION

Absorbent articles can include sanitary napkins, liners, tampons, interlabial products, and incontinence articles. There are many variations of sizes, shapes, and conformations of absorbent articles, even within a particular brand or category. Because of this diversity, consumers can have difficulty differentiating between the various types of absorbent articles, even once they have chosen a particular brand or category.

In addition, absorbent articles such as sanitary napkins have generally been contained in printed or marked packages. The markings on the packages have typically been used to attract the consumer's attention to the absorbent article, or to communicate a property of the article to the consumer. At times however, these communications in combination with the overall packaging of the article have failed to communicate the desired message to the consumer, or have misdirected or confused the consumer as to the type of absorbent article in the package. Typically, the package can include labeling, instructions, and/or a picture of the absorbent article. Nevertheless, by looking at the package, the consumer may still be unable to determine quickly any non-visual properties of the absorbent article. In some instances, the consumer may be unable to determine the non-visual properties of the absorbent article by the package exterior, or may have a limited amount of time to make a selection of a given absorbent article. In others, language difficulties can contribute to improper selection and usage.

Packages containing absorbent articles which communicate to the consumer the visual properties of the absorbent articles, is known in the art. The visual properties of the absorbent articles are communicated to the consumer using words or phrases on packages of absorbent articles such as long, regular, wings or multicolored. Additionally, pictures, diagrams and drawings on packages of absorbent articles are used to communicate the visual properties of absorbent articles to consumers. A still further method that is used to communicate visual properties of absorbent articles is through the use of transparent windows or cut outs in the packages. The windows or cut outs allow the consumer to see the visual properties of the absorbent articles.

However, the non-visual properties of absorbent articles such as flexibility, scent, or softness have not been communicated to the consumers through the use of images on packaging. Additionally, consumers need the ability to distinguish one package of absorbent articles with non-visual properties from other packages of absorbent with either different non-visual properties or those without non-visual properties.

It would be desirable to have an array of packages wherein each package has non-visual indicators located thereon to convey to a consumer a non-visual property of the absorbent articles contained therein, in relation to other packages within the array.

### SUMMARY OF THE INVENTION

An array of packages is provided which comprises a first package, said first package having disposed therein a first absorbent article having a non-visual property; a first non-visual property indicator disposed on said first package, said first non-visual property indicator being an image depicting said first absorbent article; a second package, said second package having disposed therein a second absorbent article having a non-visual property; a second non-visual property indicator disposed on said second package, said second non-visual property indicator being an image depicting said second article; and; wherein said first non-visual property indicator differs from said second non-visual property indicator to communicate the non-visual properties of said first absorbent article with respect to the non-visual properties of said second absorbent article.

An array of packages is provided which comprises a first package having a non-visual property, said first package having disposed therein a first absorbent article; a non-visual property indicator disposed on said first package, said non-visual property indicator being an image; said non-visual property indicator depicts a non-visual property of said first absorbent article; a second package, said second package having disposed therein a second absorbent article.

A package is provided which comprises a package having disposed therein an absorbent article; a non-visual property indicator disposed on said package, said non-visual property indicator being an image; said non-visual property indicator depicts a non-visual property of said absorbent article.

### BRIEF DESCRIPTION OF THE DRAWINGS

FIG. 1 is a perspective view of one embodiment of a package of the present invention.

FIG. 2 is a perspective view of one embodiment of a package of the present invention.

FIG. 3 is a perspective view of one embodiment of a package of the present invention.

FIG. 4 is a perspective view of one embodiment of the present invention.

### DETAILED DESCRIPTION OF THE INVENTION

The present invention relates to an array of packages of absorbent articles wherein at least one package has a non-visual property indicator located thereon. The non-visual indicator located on each package of the array, communicates to the consumer a non-visual property of the absorbent articles contained therein. Therefore, by viewing an array of packages with at least one package in the array having a non-visual property indicator, a consumer is able to determine a non-visual property of an absorbent article contained in at least one package, in relation to other packages of absorbent articles in the array.

As used herein, by array of packages is meant more than one package in a line-up of consumer products. The consumer products can be, and usually are, branded products in which the packages in the array of packages exhibit a common brand name. For example, the ALWAYS® sanitary napkin brand includes an array of different products, such as Ultra, Maxi, Regular, Super, Overnight, and the like, that are usually marketed as an array of packages at a point of sale. In one embodiment, an array of packages is a plurality of packages, each labeled with the same brand name. In certain embodiments there can be individual packages, having a non-visual property indicator, which are not in an array.

As used herein, the term “absorbent article” refers to devices that absorb and/or contain a substance, such as, body exudates. A typical absorbent article can be placed against or in proximity to the body of a wearer to absorb and contain various body exudates. Examples, of absorbent articles include feminine hygiene articles, and baby diapers.

As used herein, the term “feminine hygiene article” refers to articles such as, disposable absorbent articles that can be worn by women for menstrual and/or light incontinence control, such as, sanitary napkins, tampons, interlabial products, incontinence articles, and pantiliners. As used herein, a feminine hygiene article can include any associated wrapping or applicator that typically can be associated with the feminine hygiene article. For example, a feminine hygiene article can be a tampon that may or may not include an applicator and/or can be a sanitary napkin that may or may not include a wrapper, such as, a wrapper that individually encloses a single sanitary napkin.

As used herein, the term “sanitary napkin” refers to any absorbent structure such as, for example an absorbent mass that can be placed against or in close proximity to the pudendal region for absorbing menses or urine. Examples of sanitary napkins are those manufactured by The Procter & Gamble Company of Cincinnati, Ohio as: ALWAYS® Pantiliners with DriWeave® manufactured according to U.S. Pat. Nos. 4,324,246; 4,463,045; and 6,004,893; ALWAYS® Ultrathin Slender Maxi with Wings manufactured according to U.S. Pat. Nos. 4,342,314, 4,463,045, 4,556,146, B1 4,589,876, 4,687,478, 4,950,264, 5,009,653, 5,267,992, and Re. 32,649; ALWAYS® Regular Maxi; ALWAYS® Ultra Maxi with Wings; ALWAYS® Maxi with Wings; ALWAYS® Ultra Long Maxi with Wings; ALWAYS® Long Super Maxi with Wings; and ALWAYS® Overnight Maxi with Wings, each aforesaid publication being incorporated by reference herein.

As used herein, the term “image” refers to any type of mark, figure, geometric shape, picture, illustration, symbol, icon, pattern, or any other indicia on a package of within an array of packages having a purpose of communicating to a consumer something about the absorbent articles contained in the package. The image can be made in any suitable manner, such as, by printing, embossing, transferring, stamping, debossing, etching, or any combination thereof.

The non-visual property indicators of the present invention can be any image suitable for visually communicating to a consumer, a non-visual property of the absorbent articles contained in a package. A non-visual property of an absorbent article is a property that is either not visibly apparent to a consumer, such as scent, odor absorption, or odor neutralization or is not visibly apparent to a consumer prior to the use of the absorbent article, for example flexibility, softness, or smoothness. In certain embodiments, the non-visual property indicators of the present invention, can be any image suitable for visually communicating to a consumer, a non-visual property of an absorbent article contained in a package, in relation to other absorbent articles contained in other packages within an array of packages, which either do not have the non-visual property, or have a differing non-visual property.

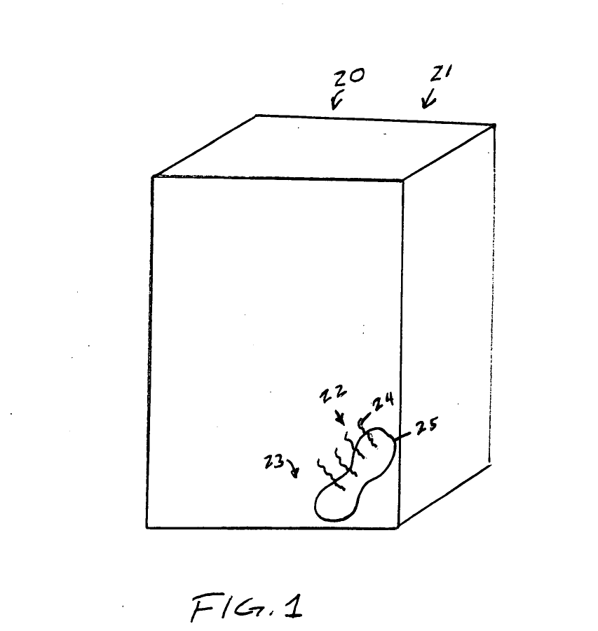

Non-visual property indicators can include any image that communicates a non-visual property of the absorbent article to a consumer. For example, as shown in FIG. 1 non-visual property indicators 22 of absorbent articles could include images indicating the absorbent article has a scent or odor, for example, to indicate scent, the image can be a sanitary napkin 23 having undulating lines 24 bisecting the sanitary napkin's perimeter 25. The non-visual property indicator 22 can be placed on the exterior surfaces 21 of a package 20.

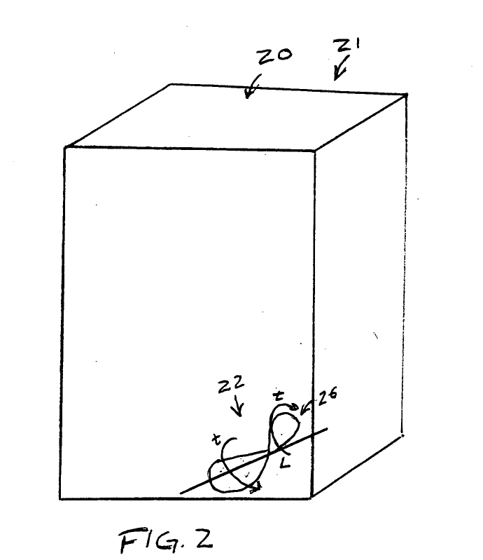

Similarly, as shown in FIG. 2, to communicate to a consumer the flexibility of an absorbent article such as a sanitary napkin, non-visual property indicators 22 of absorbent articles could include a sanitary napkin 26 wherein the opposing ends of the sanitary napkin 26 are rotated in opposing directions “t” along a longitudinal axis “L”.

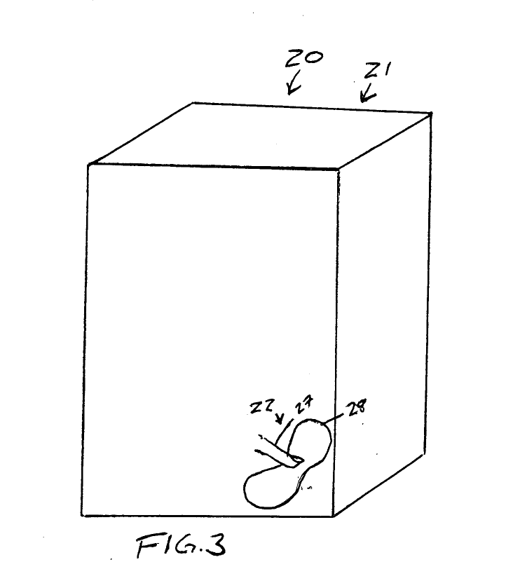

Further, as shown in FIG. 3, to communicate to a consumer the softness of an absorbent article such as a sanitary napkin, non-visual property indicators 22 of absorbent articles could include the tip of a human finger 27 touching the surface of an absorbent article 28.

In certain embodiments there may be more than one non-visual property indicator on a package. In certain other embodiments the non-visual property indicator can differ between packages of absorbent articles in an array. For example, in an array there could be non-visual indicators on packages, communicating to a consumer that at least one of the packages contains absorbent articles having a non-visual property in relation to other packages of absorbent articles in the array, that do not have the non-visual property.

In certain embodiments, the package can include at least one non-visual property indicator, wherein the absorbent articles disposed within the package can be viewed by the consumer, such as, for example, through a window or other opening on the package. The absorbent articles can be viewable by the consumer at any suitable location on the package, such as, for example, from the front of the package and/or from any other side typically viewed by the consumer. In certain embodiments, a window can be situated close to the location on the package of the non-visual property indicators.

The non-visual property indicator can be located at any suitable location on the package. In certain embodiments, the non-visual property indicator can be located on the front surface of the package, such as a surface of the package that typically can be viewed first by a consumer in a retail environment. In certain embodiments, the non-visual property indicator can be located on a secondary packaging enclosing the package. Alternatively, or in addition, the non-visual property indicator can be located somewhere other than on the package. For example, in certain embodiments, the non-visual property indicator can be located on the wrapper of an absorbent article such as a tampon or sanitary napkin.

A package can be any suitable size or shape. The package can be made of any suitable material. In certain embodiments, the package can comprise inflexible materials, such as, for example, cardboard, paperboard, cartonboard, chipboard, plywood, SBS, metal, plastic, paper, card stock, fabric, ceramic, polymer, natural or synthetic fibers, webs, mesh, screen, wood, composite, mixtures or combinations thereof, or any other suitable material. Alternatively, or in addition, the package can be made of a flexible material, such as, blown or cast film in a blend of low density polyethylene and linear low density polyethylene, metallocenes, ethylene vinyl acetate, surlyn, polyethylene terephthalate, biaxally oriented polypropylene, nylon, combinations thereof, or any other suitable material.

The package can include at least one window, such as, a region of transparency. As used herein, “transparency” means having the property of transmitting light without substantial scattering so that items lying beyond can be visible to a person. The window can comprise a transparent covering, such as, for example, a transparent film. In addition, or alternatively, the window can comprise an opening in the package that does not include a covering. In certain embodiments, the window can comprise a transparent covering that can be clear, translucent, tinted and/or opaque. The transparent covering can be tinted in any suitable color or combination of colors. The transparent covering can also, or alternatively, be printed and/or embossed. In certain embodiments, the window can be images that depict and/or give the perception of a window, such as, images that depict a region of transparency and/or an opening.

The window can be disposed on at least one side of the package, such as, the front, the back, and/or sides. In certain embodiments, the window can be disposed on more than one side. The window can be at any location on the side and can comprise any appropriate amount of the package side, such as, for example, from about 5% to about 95% of the side, such as, for example, about 5% to about 75%, about 5% to about 50%, about 5% to about 25%, or any other amount of the side. In certain embodiments, absorbent articles with a non-visual indicator disposed thereon can be positioned adjacent the window such that at least a portion of the absorbent articles and/or non-visual property indicator, can be visible through the window. In certain embodiments the wrapper of an absorbent article with a non-visual indicator disposed thereon can be positioned adjacent the window such that at least a portion of the wrapper of the absorbent article and/or non-visual property indicator, can be visible through the window.

Any absorbent article or combination of absorbent articles can be disposed in the package. In certain embodiments, the absorbent articles can be individually wrapped prior to being disposed within the package. In certain embodiments, the package can contain a single absorbent article.

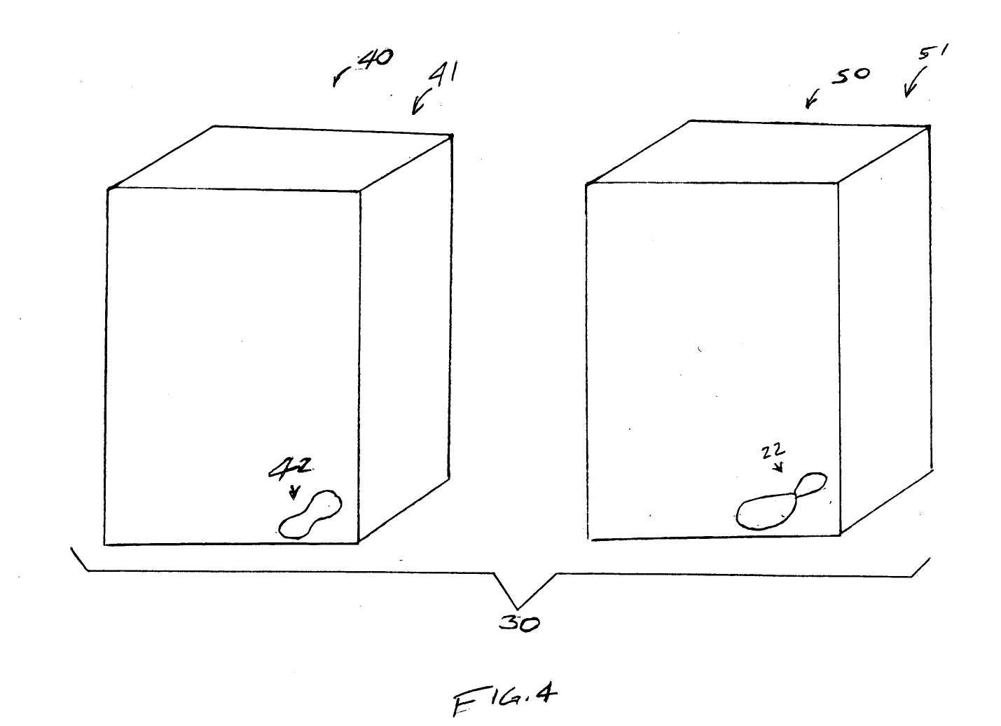

FIG. 4 shows an array 30 of packages 40, 50 containing sanitary napkins, with one package 40 without a non-visual property indicator 42 on the exterior surface 41 of the package 40 and one package 50 with a non-visual property indicator 52 disposed on the package's 50 exterior surface 51. The non-visual property indicator 52 of package 50 communicates to a consumer the flexibility of the sanitary napkins contained in the package 50 in relation to the sanitary napkins contained in the package 40 without the non-visual property indicator 22. The non-visual property indicator 52 of package 50 communicates to a consumer the non-visual property of flexibility by showing a sanitary napkin with its opposing ends twisted in opposite directions along the longitudinal axis of the sanitary napkin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of packages, said array comprising:
a first package, said first package having disposed therein a first absorbent article having a non-visual property;
a first non-visual property indicator disposed on said first package, said first non-visual property indicator being an image depicting said first absorbent article;
a second package, said second package having disposed therein a second absorbent article having a non-visual property;
a second non-visual property indicator disposed on said second package, said second non-visual property indicator being an image depicting said second article; and;
wherein said first non-visual property indicator differs from said second non-visual property indicator to communicate the non-visual properties of said first absorbent article with respect to the non-visual properties of said second absorbent article.

2. The array of claim 1 wherein there is more than one first or second non-visual property indicator disposed on the first or second package, respectively.

3. The array of claim 1 wherein the first and second non-visual property indicator is an image selected from the group consisting of a mark, figure, geometric shape, picture, illustration, symbol, icon, pattern, and any combination thereof.

4. The array of claim 1 wherein the first and second non-visual property indicators comprise different images.

5. The array of claim 1 wherein the non-visual property of said first or second absorbent article is selected from the group consisting of scent, softness, flexibility, odor absorption, odor neutralization, smoothness and combinations thereof.

6. The array of claim 1 wherein the first and second absorbent article is selected from the group consisting of sanitary napkins, tampons, interlabial products, incontinence articles, liners, and baby diapers.

7. An array of packages, said array comprising:
a first package, said first package having disposed therein a first absorbent article;
a non-visual property indicator disposed on said first package, said non-visual property indicator being an image;
said non-visual property indicator depicts a non-visual property of said first absorbent article;
a second package, said second package having disposed therein a second absorbent article.

8. The array of claim 7 wherein there is more than one non-visual property indicator disposed on the first package.

9. The array of claim 7 wherein the non-visual property indicator is an image selected from the group consisting of a mark, figure, geometric shape, picture, illustration, symbol, icon, pattern, and any combination thereof.

10. The array of claim 7 wherein the non-visual property of said first absorbent article is selected from the group consisting of scent, softness, flexibility, odor absorption, odor neutralization, smoothness, and combinations thereof.

11. The array of claim 7 wherein the first and second absorbent article is selected from the group consisting of sanitary napkins, tampons, interlabial products, incontinence articles, liners, and baby diapers.

12. A package comprising:
said package having disposed therein an absorbent article;
a non-visual property indicator disposed on said package, said non-visual property indicator being an image;
said non-visual property indicator depicts a non-visual property of said absorbent article.

13. The package of claim 12 wherein there is more than one non-visual property indicator disposed on the package.

14. The package of claim 12 wherein the non-visual property indicator is an image selected from the group consisting of a mark, figure, geometric shape, picture, illustration, symbol, icon, pattern, and any combination thereof.

15. The package of claim 12 wherein the non-visual property of said absorbent article is selected from the group consisting of scent, softness, flexibility, odor absorption, odor neutralization, smoothness, and combinations thereof.

16. The package of claim 12 wherein the absorbent article is selected from the group consisting of sanitary napkins, tampons, interlabial products, incontinence articles, liners, and baby diapers.
